(19) United States
(12) Patent Application Publication
(10) Pub. No.: US 2022/0134036 A1
(43) Pub. Date: May 5, 2022

(54) BITE BLOCK

(71) Applicant: Innovation Ventures IP at UKHS, LLC, Westwood, KS (US)

(72) Inventor: Alan Keller, Westwood, KS (US)

(21) Appl. No.: 17/520,036

(22) Filed: Nov. 5, 2021

Related U.S. Application Data
(60) Provisional application No. 63/110,076, filed on Nov. 5, 2020.

(51) Int. Cl. A61M 16/04

(52) U.S. Cl. CPC A61M 16/0493; A61M 16/0497

(57) ABSTRACT

Disclosed herein are systems, designs, and methods for a bite block and for using a bite block or surgical mouth guard. The bite block can accept various tubes used during intubation. The bite block also provides passage that allows a patient to be intubated or to breath while using a bite block. The bite block may also mitigate migration of tubes used during intubation.

### CROSS-REFERENCE TO RELATED APPLICATIONS

This application claims the benefit of the U.S. Provisional Application Ser. No. 66/110,076, entitled “Bite Block,” filed Nov. 5, 2020, the contents of which are incorporated by reference herein, for all purposes, in its entirety.

### FIELD OF THE INVENTION

The present disclosure is broadly concerned and relates to the field of medical devices used in surgical procedures. More particularly, the present invention relates to a dental guard which is designed to distribute and dissipate both direct pressure and shear forces from the patient's teeth and jaw during surgical procedures where a patient may be intubated.

### BACKGROUND

This disclosure relates generally to a bite block or dental guard that is used for the prevention of oral trauma and obstructed ventilation in anesthetized and intubated patients, wherein the device is configured to be accepted and held in the mouth of a patient, and wherein the bite block has one or more apertures for receiving and holding various tubes or other instruments or surgical devices used during surgery.

Current standards of practice for the prevention of oral trauma and obstructed ventilation in the anesthetized and intubated patients in the prone position is the use of cloth bite blocks or other makeshift devices. Most bite blocks used for either gastrointestinal scopes or transesophageal echocardiography scopes keep the mouth open by putting pressure on the front teeth. Such bite blocks are not acceptable for use during spine surgery or if a patient needs to be cardioverted or revived because the bite block will not safely prevent oral trauma from a provoked biting force. Due to no current products on the market that allow for the uniform support of a patients mouth and teeth during a procedure, medical personal routinely create current improvised bite blocks made with gauze rolled and taped into a substantially cylindrical configuration. The gauze bite blocks are then inserted bilaterally in a patient's oropharynx, specifically between the molars. The gauze bite block is commonly inserted after the patient's airway is secured by inserting an endotracheal tube. Mouthguards used for sports could be proposed for use as bite blocks; however, sports mouthguards have several shortcomings making them undesirable for use as a bite block for use during surgical and other medical procedures. Alternatively, another type of bite block currently used include cylinders that may be formed from plastic or other polymers but still have several shortcomings.

Currently, there is no standardization for how bite blocks are made or used. For example, some users may roll a single sheet of gauze to form a gauze-based bite block, while other users roll more than a single sheet of gauze. Additionally, the size of gauze sheets may vary between users where some users utilize 4×4 sheets and other users may use larger or smaller sheets. Non-standardization means the medical personnel does not follow a specific procedure and other factors such as the type and amount of tape used for rolling the gauze create a non-uniform bite block from procedure to procedure. Additionally, the technique used for rolling gauze may vary between users leading to some users making bite blocks that perform poorer than others. Failure to appropriately roll a bite block may also lead to tongue and lip lacerations. Alternatively, standardization in the development of a bite block to be used in anesthetized and intubated patients in the prone position has the possibility of limiting perioperative complications. For example, during surgery, damage can occur when a patient may bite down on surgical tubes or even their own tongue and lips. Traditional bite blocks and sports mouthguards would inhibit the use of surgical tubes by blocking passage to the patient's esophagus. Further, traditional bite block devices do not protect all a patient's teeth and instead only protect the rear teeth. Additionally, traditional bite blocks and sports mouthguards would inhibit insertion of a tube or instrument and do not prevent tube migration during surgical procedures because traditional bite blocks are not configured to hold tubes or for allowing a user to insert other devices while the bite block is engaged by the patient.

Various devices and methods have been proposed but all are limited and do not ensure proper placement of the bite block, or the tubes and other instruments used with the patient and also do not minimize successfully the potential for dental injury to the patient. The short comings of current devices or makeshift apparatuses include limited protection for the teeth, easy displacement of the dental device, difficulty in placement by the medical personal, and an obstruction of a user's view due to the bulky nature of the current devices.

What is needed is a bite block that accommodates and protects all a patient's teeth and remains in place during a medical procedure such as surgery. The bite block should also keep the mouth open by displacing the molars to minimize pressure on the patient's front teeth. The bite block should also provide passage for various tubes or other surgical devices used during surgery. The bite block should also mitigate migration of various tubes used during surgery.

### SUMMARY

The present application relates to a bite block and bite block system and methods for using the bite block with an intubated patient. In particular the bite block is a dental guard used during surgical procedures where a patient is intubated. The device is configured as a dental guard that is designed to reduce dental injuries and to manage endotracheal or gastric tubes or other instruments used during patient care.

The device is a bite block configured as a U-shaped member. The bite block is designed to engage and utilize the molars or back teeth of a patient, which provides a greater area to distribute the force exerted by a patient when they bite down during a procedure. The position on the molars is important to prevent contacting or inhibiting tubes or instruments used during a medical procedure. The bite block further includes cut-outs or openings specifically designed to allow for the accommodation of tubes and instruments during the procedure. The placement of such openings allows for the fixed placement of the tubes to ensure secure positioning during a procedure.

The device U-shaped member may have a first end and a second end. The bite block may have a top surface opposite a lower surface. The bite block may also have an interior side opposite an outer side. The bite block may also include a first leg and a second leg joined together by a middle section. The middle section may include a middle cutout and one or more side apertures. Each side aperture opens to allow tubes to be pressed into the side apertures from the top or bottom surface.

The bite block may be used by placing the bite block in a patient's mouth. The various tubes may be placed in the patient's mouth by placing the tubes in the bite block apertures before or after the bite block is placed in the patient's mouth.

### BRIEF DESCRIPTION OF THE DRAWINGS

FIG. 1 is a perspective view of the bite block according to one embodiment.

FIG. 2 is a front view of the bite block according to one embodiment.

FIG. 3 is a rear view of the bite block according to one embodiment.

FIG. 4 is a side view of the bite block according to one embodiment.

FIG. 5 is a side view of the bite block according to one embodiment.

FIG. 6 is a top view of the bite block according to one embodiment.

FIG. 7 is a bottom view of the bite block according to one embodiment.

FIG. 8 is a front view of the bite block according to one embodiment.

FIG. 9 is a side view of the bite block according to one embodiment.

FIG. 10 is a top view of the bite block according to one embodiment.

FIG. 11 is a perspective view in use of the bite block according to one embodiment

### DETAILED DESCRIPTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

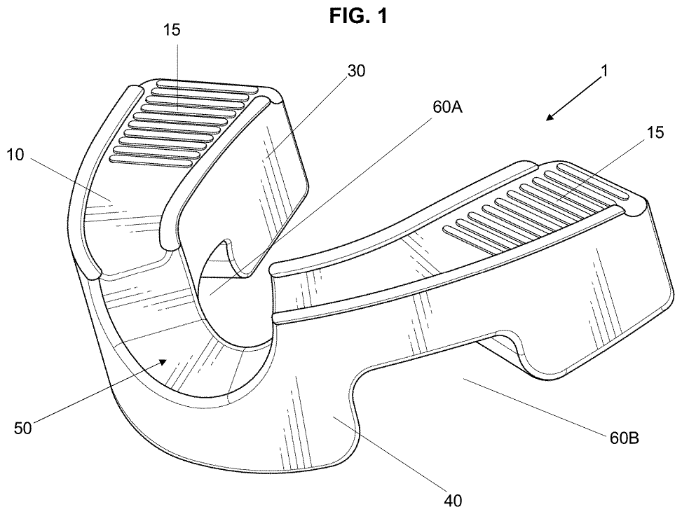

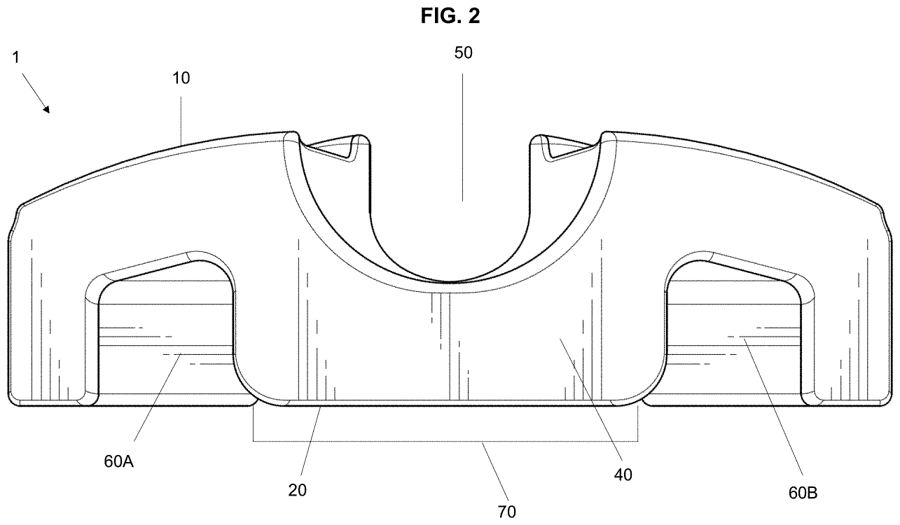

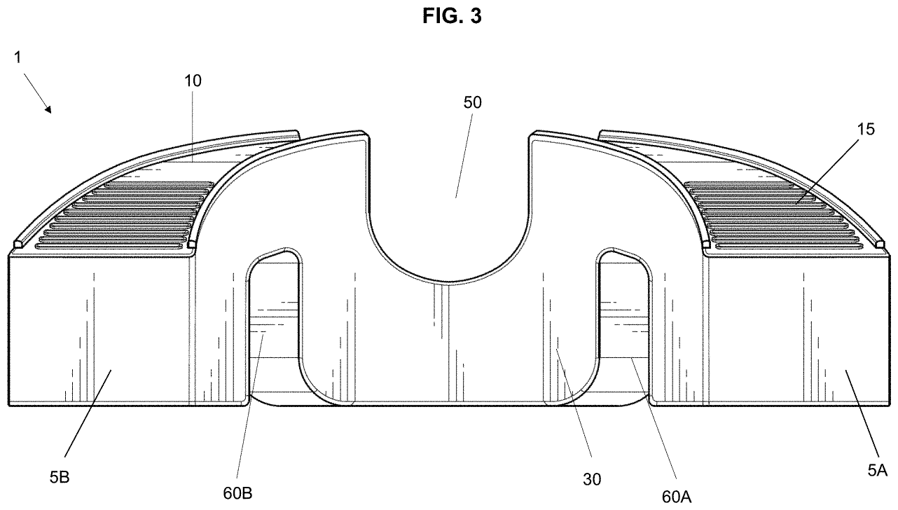

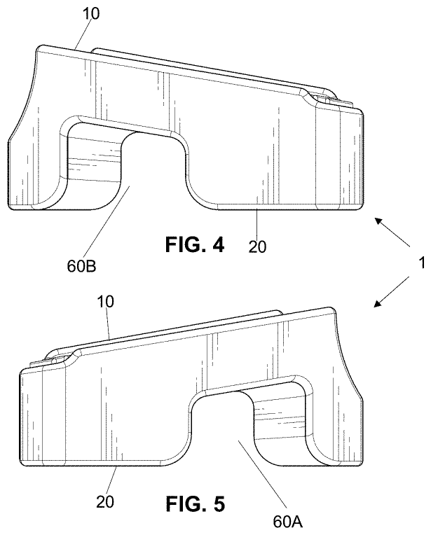

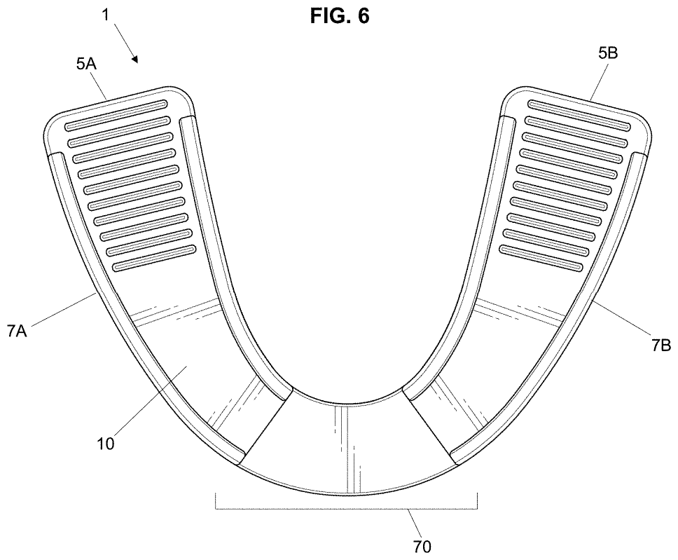

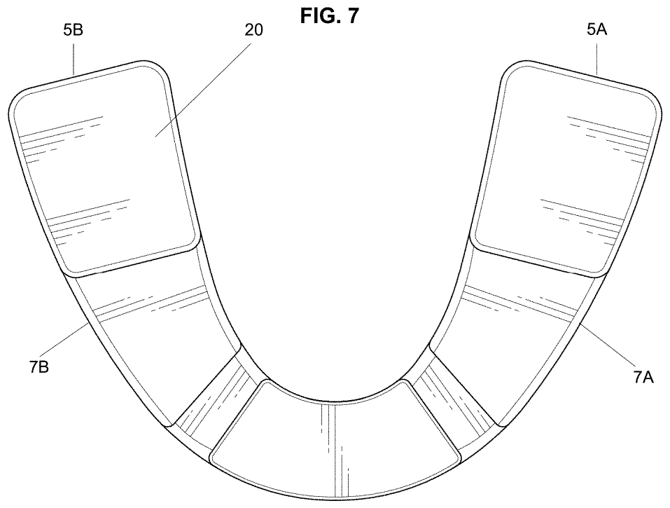

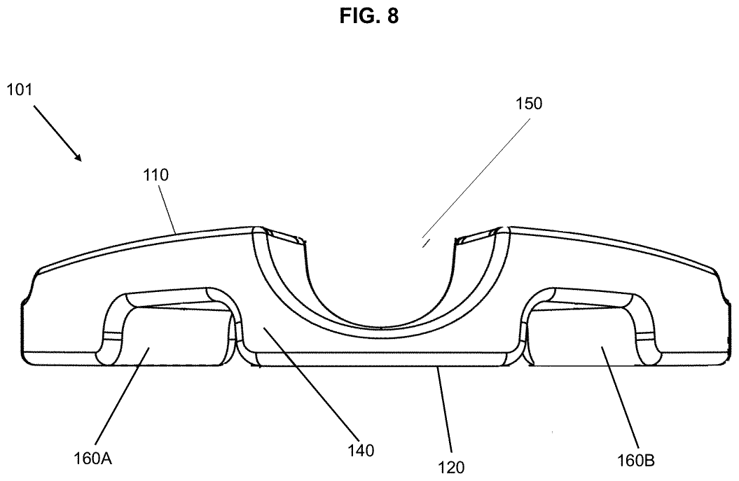

Referring now to FIGS. 1-7, there is shown an embodiment of a bite block of the present invention indicated generally by the reference numeral 1. In one embodiment, the bite block 1 has a U-shaped configuration. The bite block 1 has a first end 5A and a second end 5B. The bite block 1 has a top surface 10 opposite a lower surface 20. The bite block 1 also has an interior side 30 opposite an outer side 40. The bite block 1 further includes a first leg 7A and a second leg 7B joined by a middle section 70. The middle section 70 may include a middle cutout 50 and side apertures 60A and 60B.

As shown in FIGS. 1-7, the middle section 70 of the bite block 1 is joined to the first leg 7A and the second leg 7B. In this regard, the middle section 70 is an intermediate between the first leg 7A and the second leg 7B. The first end 5A is opposite the end of the first leg 7A that is joined to the middle section 70. Similarly, second end 5B is opposite the end of the second leg 7B that is joined to the middle section 70. In one embodiment the middle section 70 and first leg 7A and second leg 7B are composed as a single piece. In an alternative embodiment the middle section 70 and first leg 7A and second leg 7B can be more than one piece that are connected.

The top surface 10 of the bite block 1 may be smooth. In other embodiments the top surface 10 may be rough to prevent the bite block 1 from slipping while in the mouth of the patient. The top surface 10 may also have a trough to accommodate the teeth or gums of a patient. In a further embodiment, the top surface may be configured to receive a material to help in holding the user's teeth in place. The material can be a reusable sticky or tacky material or any similar material that is pliable and allows for forming around and in communication with the teeth of a patient. The material will still allow the bite block to be removed but can engage the teeth of the patient. In a further embodiment the top surface may have indicia or raised members 15 to create surface area that communicates with a user's teeth. Similarly, the bottom surface 20 of the bite block 1 may be smooth. In another embodiment, the top surface 10 and the bottom surface 20 can include any means known in the industry to create traction between the bite block and a user's teeth such as may have ridges to prevent the bite block 1 from slipping while in a patient's mouth. In other embodiments the bottom surface 20 may be rough to prevent the bite block 1 from slipping while in the mouth of the patient. The bottom surface 20 may also have a trough or indentions to accommodate the teeth or gums of a patient. In an additional embodiment the top surface 10 and the bottom surface 20 may be formed from a soft material which allows the bite block 1 to accommodate a patient's teeth. In this regard, the top surface 10 and the bottom surface 20 may be softer than the other parts of the bite block 1. The top surface 10 and the bottom surface 20 may have indicia or raised members 15 for assisting in placement of the bite block 1. The indicia may also provide friction or roughness for preventing the bite block 1 from slipping out of a patient's mouth and can be composed of material that is softer or tacky or sticky to create the connection with the patient's teeth.

The bite block 1 may have a wedge like shape where the section of the bite block 1 nearest the first end 5A and second end 5B is thinner than the middle section 70. In this regard, the bite block 70 may have dimensions that allow the bite block 1 to fit in the mouth of a patient. One of skill in the art will appreciate that the middle section 70 of the bite block 1 may have the same thickness or be thinner than the first or second ends 5A and 5B. In an alternative embodiment the bite block 1 may have varying widths to allow for the placement in the mouth of a patient.

As shown in FIGS. 1-7, the middle section 70 of the bite block 1 may have a center cutout 50. The middle cutout 50 may be used to accept an endotracheal tube or other instruments and/or items that will be used during a patient's medical procedure. As shown in FIG. 1, the center cutout 50 may have a U shape. While the center cutout 50 is preferably U shaped, the center cutout 50 may have any shape suitable for accepting tubes, including a square, triangle, pentagon, hexagon, septagon, octagon, or any other suitable shape known in the art. One of skill in the art will appreciate that the center cutout 50 may be dimensioned to accept any size of endotracheal tube known in the art. The center cutout 50 allows the user to place a tube such as an endotracheal tube in a patient's mouth without pushing the tube through a hole therefore increasing the ease of use. In this regard, the bite block 1 may be used with tubes that have a balloon or cuff because the balloon or cuff portion of the tube does not have to be pushed through an aperture. Instead, the U-shaped center cutout 50 allows a user to place or rest a tube in the center cutout 50.

The middle section 70 of the bite block 1 may also have a first side aperture 60A and a second side aperture 60B that open relative to the bottom surface 20 to create an opening slot. The first and second side apertures may be on each side of the middle cutout 50. As shown in FIGS. 1-7, the first and second aperture 60A and 60B may allow tube placement by allowing the tube to rest in the first or second aperture 60A, and 60B. In an additional embodiment, the bite block can be configured with the first and second apertures 60A and 60B open to the top surface 10. The configuration of the number of apertures on the top or bottom surface may be dependent on the tubes or instruments to be used during a procedure. The configuration of these apertures 60A and 60B are designed to be useful for placing and holding one or more tubes in the bite block 1 while a procedure is performed. Side apertures 60A and 60B may be configured to accept a gastric tube or any other tube and/or instrument or item used for intubation or the patient's procedure. One of skill in the art will appreciate there may be more than two side apertures in the bite block 1. The shape and size of the first and second side apertures can vary dependent on the user and the procedure to be performed. As shown the first and second side apertures 60A and 60B may have a square shape. The first and second side apertures 60A and 60B may have a circle shape or any shape to allow for use during a procedure. One of skill in the art will appreciate that the first and second side apertures 60A and 60B may be configured and dimensioned to accept any size of surgical tube instrument, or surgical item known in the art. The first and second side apertures allow the user to place a tube such as a gastric tube in a patient's mouth without pushing the tube through a hole. Instead, the U-shaped center cutout 50 allows a user to place or rest a tube in the center cutout 50. These configurations allow for each placement by a medical personal as well as provide comfort for a user during a procedure.

In an additional embodiment each side aperture 60A and 60B may have a separable popout section disposed within the side aperture. The center cutout 50 may also have a separable popout section disposed within the center cutout 50. The separable popouts provide the user the ability to determine the number or size of apertures needed for a procedure. A user may remove the popout sections according to the desired aperture configuration. In this regard, the bite block 1 can have various numbers of popouts for a desired configuration and is therefore customizable by the medical personal for use with the desired procedure.

The bite block 1 can also include a configuration that allows for the acceptance of a strap or other holding mechanism. This holding mechanism, such as a strap is designed help hold the bite block in place while the procedure is being perform. Alternatively, the strap can be used to also help hold tubes or instruments being used in place to minimize harm to a patient or disruption to the procedure.

The bite block 1 may be formed from a resilient material that has strength to withstand an evoked biting response. The material should also be soft enough to prevent damage to the gums, lips, teeth, if a biting response is evoked. The bite block 1 material has enough strength to withstand and resist biting force, preventing a patient from biting down on a tube placed through a side aperture 60A or 60B or center cutout 50 in the bite block 1. The bite block 1 may be formed from a pliable material such as rubber, polyvinylidene fluoride or polyvinylidene difluoride (PVDF), or other polymers known in the art. In further embodiments the bite block may be formed from more than one type of material to allow for a more pliable outer surface with a more ridged inner configuration to withstand a biting response. The more pliable outer surface can be configured to allow for a user's teeth be held securely and communicate with the bite block. In some embodiments the bite block 1 may be placed in patients that are imaged using MRI. Bite blocks used during MRI may be formed from a non-ferrous material. The bite block 1 may be translucent, allowing a user to see through the bite block 1. The bite block 1 may also be formed from a radiolucent material to allow for imaging. In one embodiment, the bite block 1 is single use. Embodiments of the bite block 1 may be compostable. In compostable embodiments, the bite block 1 may be formed from a compostable material known in the art. In an additional embodiment the bite block 1 may be reusable. Reusable embodiments of the bite block 1 may be formed from a material suitable for reprocessing. Reusable bite blocks 1 may be formed from silicone or any other material that can withstand reprocessing or sterilization.

In an additional embodiment the bite block 1 may be formed from separable sections allowing the bite block 1 to be customizable. In some embodiments, the legs 7A and 7B are separable from the middle section 70. In other embodiments each of the legs 7A and 7B may be configured to be separated into sub pieces allowing the legs 7A and 7B to be shortened depending on the desired length. In this regard, the bite block 1 may be separable and/or configured with score lines to allow for size customization. In this regard, the bite block 1 may be customizable according to the patient's mouth size, shape, and number of teeth.

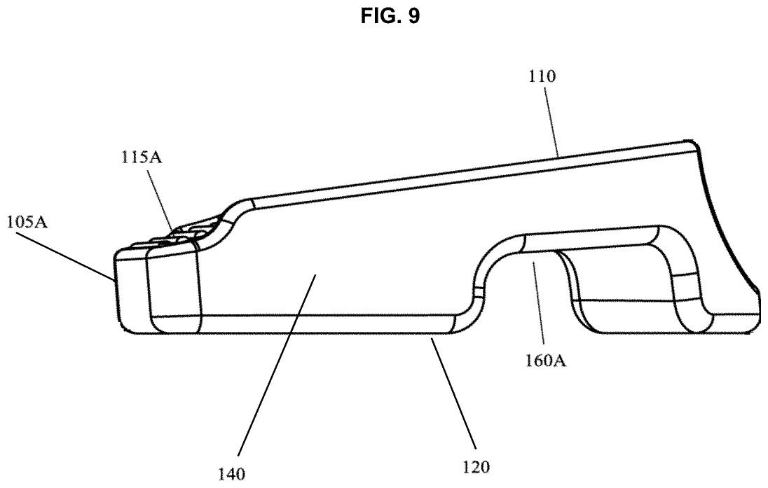

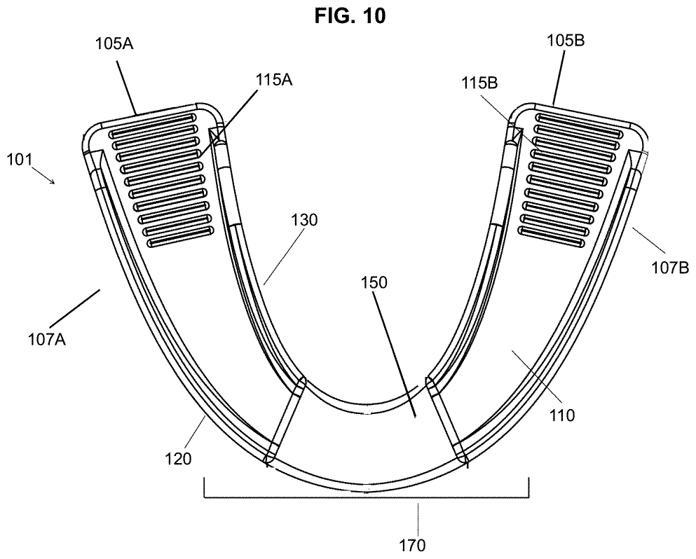

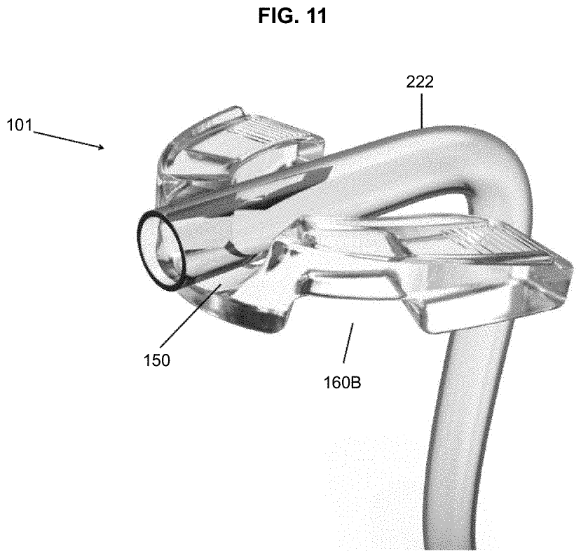

As shown in FIGS. 8-11, an additional embodiment of a bite block 101 is shown. In this embodiment, the middle section 170 of the bite block 1 or may also have a first side cutout 160A and a second side cutout 160B. The first and second side cutouts may be on each side of the middle cutout 150, with the configuration creating an opening slot. As shown in FIG. 8, the first side cutout 160A and second side cutout 160B may open at the bottom surface 120 of the bite block 101. In some embodiments the first side cutout 160A and second side cutout 160B allow tube placement by pushing one or more tubes into the first and second side cutouts. The first and/or second side cutouts may also be on the top surface 120. Side cutouts 160A and 160B may be configured to accept a gastric tube or any other tube and/or instrument or item used for intubation or the patient's procedure. One of skill in the art will appreciate there may be more than two side cutouts in the bite block 101. As shown in FIG. 8 the first and second side cutouts 160A and 160B may have a U shape. The first and second side cutouts 160A and 160B may have a circle shape or any shape to allow for use during a procedure. One of skill in the art will appreciate that the first and second side cutouts 160A and 160B may be configured and dimensioned to accept any size of surgical tube instrument, or item known in the art.

The bite block 101 has a first end 105A and a second end 105B. The bite block 101 has a top surface 110 opposite a lower surface 120. The bite block 101 also has an interior side 130 opposite an outer side 140. The bite block 1 further includes a first leg 107A and a second leg 107B joined together by a middle section 170. The middle section 170 may include a middle cutout 150 and side apertures 160A and 160B.

As shown in FIGS. 8-10, the middle section 170 of the bite block 101 is joined to the first leg 107A and the second leg 107B. The first end 105A is opposite the end of the first leg 107A that is joined to the middle section 170. Similarly, second end 105B is opposite the end of the second leg 107B that is joined to the middle section 170.

As shown in the figures, the top surface 110 may be rough to prevent the bite block 101 from slipping while in the mouth of the patient. The top surface 110 may be rough. The entire bottom surface 120 may be rough. As shown the top surface 110 of the first leg 107A and the second leg 107B may have a first ridged section 115A and a second ridged section 115B to prevent the bite block 101 from slipping while in a patient's mouth.

As shown in the figures, the middle section 170 of the bite block 101 may have a center cutout 150. The middle cutout 150 may be used to accept an endotracheal tube 222 or other instruments and/or items that will be in used during a patient's procedure. As shown in FIG. 8, the center cutout 150 may have a U shape.

The first and second side apertures 60A and 60B or the first and second side cutouts 160A and 160B may have a cross section that allows the cutout to accept and hold tubes with various diameters. In some cases the first and second side apertures 60A and 60B or first and second side cutouts 160A and 160B may be configured to accept tubes with a diameter of 10 mm, while in other cases the first and second side apertures 60A and 60B or the first and second side cutouts 160A and 160B are configured to accept tubes with a diameter of 4 mm, 5 mm, 6 mm, 7 mm, 8 mm, 9 mm, 11 mm, 12 mm, 13 mm, 14 mm, 15 mm, 16 mm, 17 mm, 18 mm, 19 mm, 20 mm, or greater than 20 mm.

The center aperture 50 or center cutout 150 may have a cross section that allows the center aperture 50 or center cutout 150 to accept and hold tubes with various diameters. In some cases the center cutout 150 may be configured to accept tubes with a diameter of 10 mm, while in other cases the center cutout 150 are configured to accept tubes with a diameter of 4 mm, 5 mm, 6 mm, 7 mm, 8 mm, 9 mm, 11 mm, 12 mm, 13 mm, 14 mm, 15 mm, 16 mm, 17 mm, 18 mm, 19 mm, or 20 mm.

The bite block 1, 101 may be formed from a single piece of material. In some embodiments, the bite block may be two, three, four, or more pieces joined together. By non-limiting example, the first and second legs 7A and 7B, 107A and 107B may be subpart pieces joined to the middle section 70, 170 by melting the pieces together. Each section or piece may be formed using injection molding. One of skill in the art will appreciate that the bite block may be formed using any manufacturing technique known in the art. One of skill in the art will appreciate that the bite block 1 may be formed of any biocompatible material known in the art. The bite block 1, 101 may be different sizes based on the user.

The bite block 1 may have different sizes for accommodating different mouth sizes. By way of non-limiting example, the bite block 1 may be small, medium, or large. In various embodiments, the legs 7A, 107A and 7B, 107B may have different lengths according the size of the patient's mouth. Similarly, the middle section 70, 170 may have different lengths according the size of the patient's mouth. Additionally, the thickness of the bite block 1, 101 may vary depending on the size of the patient's mouth.

The bite block 1, 101 may be used by an anesthetized patient. In some embodiments, the bite block 1, 101 may be placed in a patient's mouth after the patient is anesthetized and intubated. Next, an endotracheal tube may be prepared for deployment by removing an endotracheal tube connector. The endotracheal tube 222 may then be placed or pushed into the center cutout 50, 150. The endotracheal tube connector may then be reattached to the endotracheal tube. The endotracheal tube connector may be reattached to the endotracheal tube. The patient may then be connected to an anesthesia breathing circuit. An endotracheal tube pilot balloon may then be received by the first or second side aperture 60A or 60B. In some embodiments, an endotracheal tube pilot balloon may be threaded or pushed through or into the first or second side cutouts 160A and 160B. One of skill in the art will appreciate that the pilot balloon, the endotracheal tube 222, or any other tube used with the bite block 1, 101 may be placed in the middle cutout 50, 150 or either side aperture 60A or 60B, or either side cutout 160A or 160B. The endotracheal tube 222 may then be secured to the patient using tape, other adhesives, or any other traditional means for securing the tube to a patient. Because the bite block 1, 101 may be used with patients in a prone position, the user may place a strap around the posterior neck/head of the patient and affix the strap to the side protuberances on the bite block 1, 101. Attaching a strap to the side protuberances may aid the bite block 1, 101 in remaining in place when the patient is turned prone. One of skill in the art will appreciate that the bite block 1, 101 may remain in place without the assistance of a strap. Next, the practitioner may insert other monitoring devices, including temperature probes, through the side apertures of the bite block 1, 101. As the patient emerges from anesthesia, the user may leave the bite block in place until or after the endotracheal tube 222 is removed. The side apertures 60A or 60B or side cutouts 160A or 160B may be used for accessing the patient's mouth to with suction devices to remove oral secretions.

It is believed that the present disclosure and many of its attendant advantages will be understood by the foregoing description, and it will be apparent that various changes may be made in the form, construction, and arrangement of the components without departing from the disclosed subject matter or without sacrificing all its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Those skilled in the art will appreciate that variations from the specific embodiments disclosed above are contemplated by the invention. Many variations, modifications, additions, and improvements are possible. More generally, embodiments in accordance with the present disclosure have been described in the context of implementations. Functionality may be separated or combined in blocks differently in various embodiments of the disclosure or described with different terminology. These and other variations, modifications, additions, and improvements may fall within the scope of the disclosure as defined in the claims that follow.

What is claimed is:

1. A bite block formed to protect the teach when used a medical procedure comprising:
a U-shaped member, the U-shaped member comprising:
a top surface
a bottom surface opposite the top;
a first end;
a second end;
a first leg;
a second leg;
a middle section, wherein the middle section is between the first and second legs;
a central cutout in the middle section; and
at least one or more side apertures.

2. The bite block of claim 1, wherein the first leg and the second leg include at least one side aperture configured to receive a tube or medical instrument.

3. The bite block of claim 1, wherein the first leg and second leg are separable from the middle section.

4. The bite block of claim 1 further comprising separable popout sections disposed within the first aperture, the second aperture, and the center cutout.

5. The bite block of claim 2, wherein the side aperture opens to the bottom surface of the bite block to create an opening slot.

6. The bite block of claim 2, wherein the side aperture opens to the top surface of the bite block to create an opening slot.

7. The bite block of claim 1, wherein the bite block further includes a trough or channel on the top surface to accommodate placement of the user's teeth.

8. The bite block of claim 1, wherein the bite block further includes grip mechanisms that can include indica, ridges or the combination thereof to create a hold on the user's teeth.

9. The bite block of claim 8, wherein the grip mechanism is composed of a pliable material that is softer than the material formed to create the body of the bite block.

10. The bite block of claim 1, wherein the bite block is formed from a single piece of material.

11. The bite block of claim 1, wherein the bite block is formed from more than one piece of material.

12. The bite block of claim 1, wherein the first leg and the second leg have a thinner height than the middle section to create a wedge shaped configuration for the bite block.

13. The bite block of claim 1, wherein the first leg and second leg include score lines to allow for accommodation to various sizes.

14. The bite block of claim 1, wherein the bite bock further includes a strap mechanism to hold the bite block in place.

15. A method of using the bite block comprising:
placing the bite block in a patient's mouth; wherein the bite block includes a U-shaped member comprised of a top surface, a bottom surface, a first end, a second end, a first leg configured with a side aperture, a second leg configured with a side aperture, and a middle section, wherein the middle section includes a central cutout configured to receive an intubation tube,
inserting at least one tube into the patient's mouth;
placing the at least one tube in communication with the central cutout configured to receive the intubation tube in the bite block.
